(19) United States
(12) Patent Application Publication
(10) Pub. No.: US 2007/0178143 A1
(43) Pub. Date: Aug. 2, 2007

(54) Methods for Altering Food Intake by Animals

(76) Inventors: Ryan Michael Yamka, Topeka, KS (US); Kim Gene Friesen, Topeka, KS (US); Thomas Gordon Forster, Topeka, KS (US); Lauren Kats, Topeka, KS (US)

Correspondence Address: COLGATE-PALMOLIVE COMPANY, 909 RIVER ROAD, PISCATAWAY, NJ, 08855, US

(21) Appl. No.: 11/620,119

(22) Filed: Jan. 5, 2007

Related U.S. Application Data
(60) Provisional application No. 60/756,341, filed on Jan. 5, 2006.

(51) Int. Cl. A61K 47/00; A23K 1/165

(52) U.S. Cl. 424/442; 426/635

(57) ABSTRACT

The invention provides methods for altering average food intake by an animal by adjusting the balance of metabolizable cations to metabolizable anions consumed by the animal by an amount effective to alter average food intake. The invention also provides articles of manufacture for use with the methods and means for communicating information about the methods and the articles of manufacture.

This application claims benefit of U.S. Provisional No. 60/756341 filed Jan. 5, 2006 which is incorporated by reference in its entirety for all purposes.

### BACKGROUND OF THE INVENTION

1. Field of the Invention

The present invention relates generally to methods for altering food intake by an animal. The invention also relates generally to articles of manufacture for use with such methods and means for communicating information about such methods and such articles of manufacture.

2. Description of the Related Art

The regulation of food intake is an important factor in controlling weight gain of weight loss in an animal. Companion animals considered overweight and/or obese are increasing in number such that it is now estimated that in the U.S., between 25% and 40% of companion animals are considered overweight or obese. An animal is consideLed overweight if it weighs more than 10% above its ideal body weight, and obese if it weighs more thlcu 15% above its ideal body weight. A companion animal has an ideal body weight if the animal's ribs can be felt, but not seen. Obesity in companion animals is implicated in increased risk of diabetes mellitus, arthritis, pancreatitis, hepatic lipidosis, orthopedic disorders, cardiovascular disease, respiratory ailments, hip dysplasia, liver disease, gastrointestinal disorders, and skin problems. Further, some breeds of dogs and cats have particular propensities for rapid weight gain in spite of a diligent owner's attempts to curb the pet's increasing weight.

Likewise, it is often necessary or desirable to increase food intake by an animal that is glowing or underweight. Thus, animals, like humans, have been barraged with dieting schemes and exercise regimens to influence body weight. Advances have been made in the development of particular foods for controlling weight gain or weight loss in animals; however, opportunities for improvement remain such that there continues to be a need for new methods and compositions for altering food intake by animals.

### SUMMARY OF THE INVENTION

It is an object of the present invention to provide methods for altering average food intake by an animal. The methods comprise adjusting the balance of metabolizable cations to metabolizable anions consumed by the animal by an amount effective to alter average food intake.

It is another object of the invention to provide methods for controlling weight of an animal. The methods comprise adjusting the balance of metabolizable cations to metabolizable anions consumed by the animal to alter average food intake in an amount effective to influence the animal's weight.

It is another object of the invention to provide articles of manufacture comprising two or more ingredients that, when combined together and optionally with additional ingredients that are not a part of the article of manufacture, yield a composition for use in the methods of this invention. The articles of manufacture comprise a food intake altering amount of at least one ingredient comprising a metabolizable cation or metabolizable anion and at least one of (1) an ingredient comprising a different metabolizable cation or metabolizable anion; (2) one or more ingredients for consumption by an animal; (3) one or more weight-loss agents; and (5) one or more agents for promoting weight-gain.

It is a further object of this invention to provide means for communicating information about the methods mid articles of manufacture of this invention.

Other and further objects, features, and advantages of the present invention will be readily apparent to those skilled in the art.

### DETAILED DESCRIPTION OF THE INVENTION

In one aspect, the present invention provides methods for altering food intake by an animal. The term “animal” includes any animal susceptible to or suffering from obesity as well as any animal in need of weight management including weight loss, weight maintenance or weight gain. An animal is “susceptible to” a disease or condition if the animal exhibits symptoms that indicate that the animal is likely to develop the condition or disease. An animal is “suffering from” a disease or condition if the animal exhibits symptoms that are indicative that the animal has developed the condition or disease.

In some embodiments, the animal is a member of the order Carnivora. In some such embodiments, the animal is a canine, and in other such embodiments a feline. In some embodiments, the animal is a companion animal. A companion animal can be, for example, an animal of any species that is kept as a pet. A companion animal can also be an animal from a variety of widely domesticated species, for example, dogs (Canis familiaris) and cats (Felis domesticus) regardless of whether or not the animal is kept solely as a pet. Thus, companion animals include, for example, working dogs, cats kept for rodent control, as well as pet cats and dogs.

The methods of the present invention comprise adjusting the balance of metabolizable cations to metabolizable anions regularly consumed by the animal by an amount effective to alter the animal's average food intake. Generally, the balance of metabolizable cations to metabolizable anions can be determined by any means known in the art. In one embodiment, the metabolizable cations are selected from the group consisting of calcium, sodium, potassium and magnesium; and the metabolizable anions are selected from the group consisting of phosphorus, chloride and sulfur. One method for measuring the balance of metabolizable cations to metabolizable anions is to calculate the animal's dietary cation-anion balance (DCAB). DCAB is determined by calculating the cumulative amount of sodium, potassium, calcium, and magnesium regularly consumed by the animal and subtracting the cumulative amount of chloride, sulfur, and phosphorus regularly consumed by the animal. See, Baker et al., Comparative Nutrition of Cats and Dogs, Ann. Rev. Nutr. 11:239-63 (1991).

In adjusting the balance of metabolizable cations to metabolizable anions consumed by the animal, Applicants have discovered that increasing the balance of metabolizable cations to metabolizable anions will result in increased average food intake. Accordingly, decreasing the balance of metabolizable cations to metabolizable anions will result in decreased average food intake.

In some embodiments wherein the animal is need of weight gain, for example, the animal is susceptible to or suffering from low body weight, food intake may be increased by increasing the balance of metabolizable cations to metabolizable anions. In such embodiments, the balance can be increased by increasing the cumulative amount of calcium, sodium, potassium and magnesium regularly consumed by the animal relative to the cumulative amount of phosphorus, chloride and sulfur regularly consumed by the animal. For example, the balance of metabolizable cations to metabolizable anions may be increased by increasing the animal's dietary intake of at least one composition comprising calcium, sodium, potassium or magnesium. Likewise, the balance may be increased by decreasing the animal's dietary intake of at least one composition comprising chloride, phosphorus or sulfur.

In other embodiments wherein the animal is need of weight loss, for example, the animal is susceptible to or suffering from obesity, food intake can be decreased by decreasing the balance of metabolizable cations to metabolizable anions consumed by the animal. In such embodiments, the balance can be decreased by decreasing the cumulative amount of calcium, sodium, potassium and magnesium regularly consumed by the animal relative to the cumulative amount of phosphorus, chloride and sulfur regularly consumed by the animal. For example, the balance of metabolizable cations to metabolizable anions can be decreased by decreasing the animal's dietary intake of at least one composition comprising calcium, sodium, potassium or magnesium. Likewise, the balance may be decreased by increasing the animal's dietary intake of at least one composition comprising chloride, phosphorus or sulfur.

In some embodiments, the balance of metabolizable cations to metabolizable anions consumed by the animal can be adjusted by feeding the animal a composition comprising a food intake altering amount of one or more metabolizable cations or metabolizable anions. Such compositions can include food compositions. In some embodiments, the food composition comprises a dry food (i.e., a food containing from about 3 to about 11% water). In other embodiments, the food composition comprises a semi-moist food (i.e., a food containing from about 25 to about 35% water). In some embodiments, the food composition comprises a moist food (i.e., a food containing from about 60 to more than about 87% water). In some embodiments, the food composition comprises a treat, snack, supplement, or partially or fully edible toy.

In some embodiments, the balance of metabolizable cations to metabolizable anions consumed by the animal can be adjusted in conjunction with the administration of one or more weight-loss agents. The term “weight-loss agent” means any compound, composition, or drug useful for regulating weight or preventing or treating obesity. Examples include, but are not limited to, commercially available “low fat” or “low calorie” pet food compositions. Likewise, in some embodiments, the balance of metabolizable cations to metabolizable anions consumed by the animal can be adjusted in conjunction with the administration of one or more agents for promoting weight gain. The term “agent for promoting weight gain” means any compound, composition, or drug useful for increasing weight and may include, but is not limited to, commercial and/or prescription diet formulations available for nutritional supplementation.

In another aspect, the present invention provides an article of manufacture, Such as for example, a kit comprising a composition comprising a food intake altering amount of at least one metabolizable cation or metabolizable anion. In some embodiments, the kit further comprises one or more weight-loss agents or one or more agents for promoting weight gain. In some embodiments, the kit further comprises instructions for at least one of (1) feeding the composition to an animal to adjust the balance of metabolizable cations to metabolizable anions consumed by the animal, or (2) administering a weight-loss agent or a weight-gain agent in conjunction with the composition to alter an animal's average food intake.

In some embodiments, the kit comprises in separate containers in a single package or in separate containers in a virtual package, as appropriate, a food intake altering amount of at least one ingredient comprising a metabolizable cation or metabolizable anion and at least one of (1) an ingredient comprising a different metabolizable cation or metabolizable anion; (2) one or more ingredients for consumption by an animal; (3) one or more weight-loss agents; (4) one or more agents for promoting weight gain; (5) instructions for combining one or more relevant kit components to prepare a composition useful for altering average food intake; and (6) instructions for using one or more kit components to alter average food intake.

The term “single package” generally means that the components of a kit are physically associated in or with one or more containers and considered as a unit of manufacture, distribution, sale, or use. Containers include, but are not limited to, bags, boxes, bottles, shrink wrap packages, stapled or otherwise fixed components, or combinations thereof. A single package can be, for example, containers or individual food compositions physically associated such that they are considered a unit for manufacture, distribution, sale, or use. The term “virtual package” generally means that the components of a kit are associated by directions on one or more physical or virtual kit components instructing the user how to obtain additional optional components, e.g., in a bag containing one component and directions instructing the user to go to a website, contact a recorded message, view a visual message, or contact a caregiver to obtain instructions on how to use the kit. When the kit comprises a virtual package, the kit is limited to instructions in a virtual environment with one or more physical kit components.

In another aspect, the present invention provides a means for communicating information about or instructions for one or more of (1) using a balance of metabolizable cations to metabolizable anions consumed by an animal to alter average food intake, (2) admixing metabolizable cations and metabolizable anions with other ingredients and nutrients, (3) administering metabolizable cations and metabolizable anions to an animal, alone or in combination with other ingredients and nutrients, and (4) using the kits of the present invention for altering average food intake, comprising a document, digital storage media, optical storage media, audio presentation, or visual display containing the information or instructions. In certain embodiments, the communicating means comprises a document, digital storage media, optical storage media, audio presentation, or visual display containing the information or instructions. Preferably, the communication means is a displayed web site or a brochure, product label, package insert, advertisement, or visual display containing such information or instructions. Useful information or instructions include, for example, (1) information and instructions how to use a composition, method, or kit described in the present invention and (2) contact information for animal caregivers if they have a question about the invention and its uses.

In a further aspect, the present invention provides for a use of a composition that comprises a food intake altering amount of at least one ingredient selected from the group consisting of metabolizable cations and metabolizable anions to prepare a medicament. In another, the invention provides for the use of such a composition to prepare a medicament for altering food intake. Generally, medicaments are prepared by admixing a compound or composition with excipients, buffers, binders, plasticizers, colorants, diluents, compressing agents, lubricants, flavorants, moistening agents, and other ingredients known to skilled artisans to be useful for producing medicaments and formulating medicaments that are suitable for administration to an animal.

The invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise. Similarly, the words “comprise”, “comprises”, and “comprising” are to be interpreted inclusively rather than exclusively.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents, patent applications, and other references mentioned herein are incorporated herein by reference to the extent allowed by law for the purpose of describing and disclosing the compounds, processes, techniques, procedures, technology, articles, and other compositions and methods disclosed therein that might be used with the present invention. However, nothing herein is to be construed as an admission that the invention is not entitled to antedate such disclosure by virtue of prior invention.

### EXAMPLES

This invention can be further illustrated by the following example, although it will be understood that the example is included merely for purposes of illustration and is not intended to limit the scope of the invention unless otherwise specifically indicated.

### Example 1

Twenty dogs are randomly assigned to one of two groups containing 10 dogs per group. Each group receives one of two foods formulated to contain a different balance of metabolizable cations to metabolizable anions. The balance of metabolizable cations to metabolizable anion is calculated as DCAB according to conventional methods comprising the cumulative amount of sodium, potassium, calcium, and magnesium minus the cumulative amount of chloride, sulfur and phosphorus, taking into account the need to convert data for cations and anions from percentage to milliequivalents (meq) as provided in Table 2. The dogs are fed the foods for seven clays over which time the dog's average food intake is quantified.

Results showing the nutrient analysis of each food and the average food intake for each food are presented in Table 1.

TABLE 1
Analyzed Nutrient Composition of Foods Fed to Dogs^{a}

| Item | Food 1 | Food 2 |
|---|---|---|
| Crude Protein (%) | 20.2 | 19.1 |
| Crude Fat (%) | 13.0 | 14.2 |
| Crude Fiber (%) | 2.9 | 3.2 |
| Sodium (%) | 0.39 | 0.30 |
| Potassium (%) | 0.87 | 0.71 |
| Chloride (%) | 0.97 | 0.74 |
| Sulfur (%) | 0.40 | 0.33 |
| Calcium (%) | 0.63 | 0.66 |
| Magnesium (%) | 0.06 | 0.05 |
| Phosphorus (%) | 0.58 | 0.50 |
| DCAB^{b} (meq) | −107 | −23 |
| Avg. food intake (g/day) | 152 | 194 |

^{a}Dry Matter Basis
^{b}DCAB = (Sodium + Potassium + Calcium + Magnesium) − (Chloride + Sulfur + Phosphorous)

TABLE 2
Conversion Data for Calculating DCAB

| | | | | To convert % |
|---|---|---|---|---|
| | | | | to meq |
| | Molecular | | Equivalent | multiply by |
| Element | weight (g) | Valence | weight (g) | this number |
| Sodium | 23.0 | 1 | 23.0 | 434.98 |
| Potassium | 39.1 | 1 | 39.1 | 255.74 |
| Chloride | 35.5 | 1 | 35.5 | 282.06 |
| Sulfur | 32.1 | 2 | 16.0 | 623.75 |
| Calcium | 40.1 | 2 | 20.0 | 499.00 |
| Magnesium | 24.3 | 2 | 12.2 | 822.64 |
| Phosphorous | 31.0 | 1.8 | 17.2 | 581.14 |

Results indicate that the balance of metabolizable cations to metabolizable anions in the foods is directly related to average food intake. Dogs fed Food 2 having a higher balance of metabolizable cations to metabolizable anions or DCAB demonstrate an average food intake increase of 42 g/day.

In the specification, there have been disclosed typical preferred embodiments of the invention and, although specific terms are employed, they are used in a generic and descriptive sense only and not for purposes of limitation, the scope of the invention being set forth in the claims. Obviously many modifications and variations of the invention are possible in light of the above teachings. It is therefore to be understood that within the scope of the appended claims the invention may be practiced otherwise than as specifically described.

Although any compositions, methods, kits, and means for communicating information similar or equivalent to those described herein can be used to practice this invention, the preferred compositions, methods, kits, and means for communicating information are described herein.

What is claimed is:

1. A method for altering average food intake by an animal comprising adjusting the balance of metabolizable cations to metabolizable anions consumed by the animal by an amount effective to alter average food intake.

2. The method of claim 1 wherein the metabolizable cations are selected from the group consisting of calcium, sodium, potassium, and magnesium.

3. The method of claim 1 wherein the metabolizable anions are selected from the group consisting of phosphorus, chloride, and sulfur.

4. The method of claim 1 wherein average food intake is increased by increasing the balance of metabolizable cations to metabolizable anions.

5. The method of claim 4 wherein the balance is increased by increasing the cumulative amount of calcium, sodium, potassium and magnesium regularly consumed by the animal relative to the cumulative amount of phosphorus, chloride and sulfur regularly consumed by the animal.

6. The method of claim 4 wherein the balance is increased by increasing the animal's dietary intake of at least one composition comprising calcium, sodium, potassium, or magnesium.

7. The method of claim 4 wherein the balance is increased by decreasing the animal's dietary intake of at least one composition comprising phosphorus, chloride, or sulfur.

8. The method of claim 1 wherein average food intake is decreased by decreasing the balance of metabolizable cations to metabolizable anions.

9. The method of claim 8 wherein the balance is decreased by decreasing the cumulative amount of calcium, sodium, potassium and magnesium regularly consumed by the animal relative to the cumulative amount of phosphorus, chloride and sulfur regularly consumed by the animal.

10. The method of claim 8 wherein the balance is decreased by decreasing the animal's dietary intake of at least one composition comprising, calcium, sodium, potassium, or magnesium.

11. The method of claim 8 wherein the balance is decreased by increasing the animal's dietary intake of at least one composition comprising phosphorus, chloride, or sulfur.

12. The method of claim 1 wherein the animal is a companion animal.

13. The method of claim 1 wherein the animal is a dog or a cat.

14. A method for controlling weight of an animal comprising adjusting the balance of metabolizable cations to metabolizable anions consumed by the animal to alter average food intake in an amount effective to influence weight.

15. The method of claim 14 wherein the balance of metabolizable cations to metabolizable anions is increased to increase average food intake.

16. The method of claim 15 wherein the balance is increased by increasing the cumulative amount of calcium, sodium, potassium and magnesium regularly consumed by the animal relative to the cumulative amount of phosphorus, chloride and sulfur regularly consumed by the animal.

17. The method of claim 14 wherein the balance of metabolizable cations to metabolizable anions is decreased to decrease average food intake.

18. The method of claim 15 wherein the balance is decreased by decreasing the cumulative amount of calcium, sodium, potassium and magnesium regularly consumed by the animal relative to the cumulative amount of phosphorus, chloride and sulfur regularly consumed by the animal.

19. The method of claim 14 wherein the animal is a companion animal.

20. A kit suitable for altering average food intake comprising in separate containers in a single package or in separate containers in a virtual package, as appropriate, a food intake altering amount of at least one ingredient comprising a metabolizable cation or metabolizable anion and at least one of (1) an ingredient comprising a different metabolizable cation or metabolizable anion; (2) one or more ingredients for consumption by an animal; (3) one or more weight-loss agents; (4) one or more agents for promoting weight gain; (5) instructions for combining one or more relevant kit components to prepare a composition useful for altering food intake; and (6) instructions for using one or more kit components to alter food intake.

21. A means for communicating information about or instructions for one or more of (1) using a balance of metabolizable cations to metabolizable anions consumed by an animal to alter food intake, (2) admixing metabolizable cations and metabolizable anions with other ingredients and nutrients, (3) administering metabolizable cations and metabolizable anions to an animal, alone or in combination with other ingredients and nutrients, and (4) using the kits of the present invention for altering food intake, the means comprising a document, digital storage media, audio presentation, or visual display containing the information or instructions.

22. The means of claim 21 selected from the group consisting of a displayed website, brochure, product label, package insert, advertisement, displayed web site, and visual display.

23. A use of a composition that comprises a food intake altering amount of at least one ingredient selected from the group consisting of metabolizable cations and metabolizable anions to prepare a medicament for altering food intake.
